(19) United States
(12) Patent Application Publication
(10) Pub. No.: US 2024/0412819 A1
(43) Pub. Date: Dec. 12, 2024

(54) METHOD FOR IDENTIFYING ENZYME DIGESTION SITE FOR NUCLEIC ACID NICKASE

(71) Applicant: GUANGDONG GENERAL HOSPITAL, Guangzhou (CN)

(72) Inventors: Bing GU, Guangzhou (CN); Xiaoxiao LIU, Guangzhou (CN); Zixia WANG, Guangzhou (CN); Yong LING, Guangzhou (CN); Xiaozhong CHEN, Guangzhou (CN); Zhixuan ZHANG, Guangzhou (CN); Ziqing DENG, Guangzhou (CN)

(73) Assignee: GUANGDONG GENERAL HOSPITAL, Guangzhou (CN)

(21) Appl. No.: 18/512,105

(22) Filed: Nov. 17, 2023

(30) Foreign Application Priority Data
Jun. 12, 2023 (CN) 202310693196.6

(51) Int. Cl. G16B 30/10

(52) U.S. Cl. CPC G16B 30/10

(57) ABSTRACT

A method for identifying an enzyme digestion site of a nucleic acid nickase is provided. The method uses mNGS sequencing to obtain nucleic acid sequence data before and after enzyme digestion, uses Bowite2 and samtools software to analyze second-generation sequencing data, and obtains the action site of the nickase according to the depth and analysis of single-base sequencing. The method does not need to clarify the sequence of the nucleic acid to be cut, takes a short time and low budget, and thus has a good application prospect.

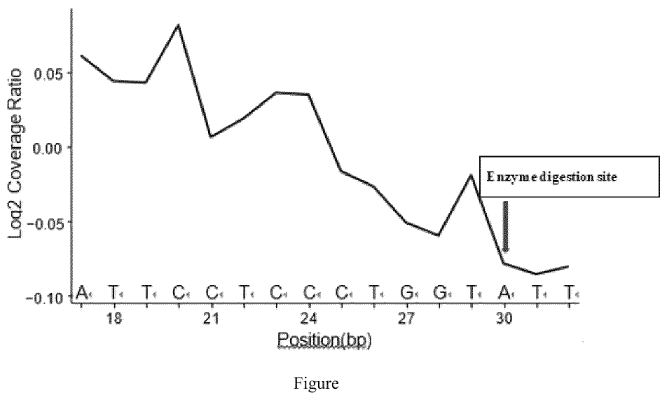

### CROSS REFERENCE TO THE RELATED APPLICATIONS

This application is based upon and claims priority to Chinese Patent Application No. 202310693196.6, filed on Jun. 12, 2023, the entire contents of which are incorporated herein by reference.

### TECHNICAL FIELD

The present invention relates to the field of biotechnology, in particular to a method for identifying an enzyme digestion site of a nucleic acid nickase.

### BACKGROUND

Nucleic acid detection is widely used in food safety, biomedical testing, environmental testing and other fields. A nucleic acid sequence-specific isothermal and polymerase chain reaction (PCR) amplification technology is a typical representative of the vigorous development of molecular diagnostics in recent years. A nickase (nicking nuclease) can effectively replace DNA double-strand endonuclease for a rapid constant temperature amplification technology, which makes the isothermal amplification technology enter a new stage of development and move towards practical application faster. The key to the use of nickases (nicking nuclease) is to clarify the specific enzyme digestion sites of nickases (nicking nuclease), which is related to the design of primers and the development of corresponding kits for subsequent applications. However, the existing method for identifying a digestion site of a nickase (nicking nuclease) needs to first clarify a nucleic acid sequence to be cut, which takes a long time and a high budget.

### SUMMARY

In view of this, the technical problem to be solved by the present invention is to provide a method for identifying an enzyme digestion site of a nucleic acid nickase.

The present invention provides a method for identifying a digestion site of a nickase. The includes the following steps:
step 1, sequencing samples before and after nickase digestion to obtain sample data 1 before nickase digestion and sample data 2 after nickase digestion;
step 2, trimming and filtering the sample data 1 and the sample data 2 respectively to obtain quality control data 1 and quality control data 2;
step 3, after aligning and sorting the quality control data 1 and the quality control data 2, obtaining a sequencing depth of a base site; and
step 4, calculating a Log 2 Coverage Ratio value according to the sequencing depth of the base site, then making a line graph, and determining the digestion site of the nickase according to the Log 2 Coverage Ratio value and the line graph.

In the identification method described in the present invention,
the Log 2 Coverage Ratio value is: taking 2 as a base, a logarithm of a ratio of the sequencing depth of the base site after enzyme digestion to the sequencing depth of the base site before enzyme digestion.

The specific formula is as follows:

Log 2 Coverage Ratio=log₂ (Depth of the sample after enzyme digestion/Depth of the sample before enzyme digestion)

A criterion for determining the digestion site of the nickase is: the lowest valley with the Log 2 Coverage Ratio value being less than 0.06 and an obvious valley fracture being the digestion site of the nickase.

In step 1 of the identification method according to the present invention, the sample is a double-stranded DNA sample.

The sequencing is mNGS next-generation sequencing.

In step 2 of the identification method according to the present invention, trimming parameters are set as follows: removing an adapter, setting -5 to 20, and setting -3 to 20.

In step 2 of the identification method according to the present invention, filtering parameters are set as follows: setting -q to 20, setting -n v 15, and setting -1 to 80.

In the identification method according to the present invention, it is necessary to remove unaligned reads after the alignment and before the sorting.

In the identification method according to the present invention, software for the alignment is bowtie2, a mode of the alignment is an end-to-end mode, and parameters of the end-to-end mode are set as: very-sensitive, setting -L to 30, setting -score-min to L, -0.6, -0.2.

In the identification method according to the present invention, the nickase is a restriction endonuclease that generates a single-strand nick in or near the specific DNA sequence.

According to the identification method described in the present invention, the digestion site of the nickase can be accurately found, and changes in parameters or steps will affect the analysis speed and accuracy of identification results. In the present invention, the first step removes the adapter, reducing the risk that it is difficult to completely remove adapter sequences after processing of the subsequent steps. Both -3 and -5 are set to 20, which removes the bases whose quality is lower than a threshold in the reads, such that the accuracy of the analysis results is better compared with other values. The alignment is performed using the bowtie2 software, selecting very-sensitive in the end-to-end mode further improves the data quality, and more accurate alignment results can be obtained in comparison with sensitive. At the same time, the digestion site of the nickase is single, and precise and rigorous quality control parameter settings reduce retained data and speed up analysis.

The present invention discloses the method for identifying an action site of the nickase. The method is to use mNGS sequencing to obtain nucleic acid sequence data before and after enzyme digestion. Bowite2 software and samtools software are adopted to analyze the second-generation sequencing data, and obtain the action site of the nickase according to the depth and analysis of single-base sequencing. The method of the present invention does not need to clarify the sequence of the nucleic acid to be cut, takes a short time and low budget, and thus has a good application prospect.

### BRIEF DESCRIPTION OF THE DRAWINGS

The FIGURE shows a linear diagram of nickase digestion sites.

### DETAILED DESCRIPTION OF THE EMBODIMENTS

The present invention provides a method for identifying an enzyme digestion site of a nucleic acid nickase, and those skilled in the art can refer to the content herein and appropriately improve process parameter implementations. In particular, it should be pointed out that all similar replacements and modifications are obvious to those skilled in the art, and they are all considered to be included in the present invention. The method and application of the present invention have been described through preferred embodiments, and relevant personnel can obviously make alterations or appropriate changes and combinations to the method and application herein without departing from the content, spirit and scope of the present invention to implement and apply the present invention.

The test materials used in the present invention are all common commercially available products, which can be purchased in the market.

The present invention is further set forth below in conjunction with an embodiment.

### Embodiment 1 A Method for Identifying an Enzyme Digestion Site of a Nucleic Acid Nickase

mNGS next-generation sequencing is performed on a nucleic acid before and after enzyme digestion, and bioinformatics methods are adopted for analysis according to the following steps. The analysis is mainly performed using fastp, and main parameters of fastp are set to -5 20 -3 20 -q 20 -n 15 -l 80, default values are adopted for other parameters, and reads that do not meet the requirements are removed:
(1) adapter filtering, -A turns off adapter trimming, default software will cut out the adapter, and if -A is set, this function is turned off, and this project uses the function of removing the adapter;
-a given an adapter sequence file, this project uses the software's own adapter library;
(2) global trimming options
-f, -trim_front1 means trimming the number of bases before read1, which is 0 by default in this project;
-F, -trim_front2 means trimming the number of bases before read2, which is 0 by default in this project;
-t, -trim_tail1 means trimming the number of bases at the tail of read1, which is 0 by default in this project;
-T, -trim_tail2 means trimming the number of bases at the tail of read2, which is 0 by default in this project;
-b, -max_len1 means, if read1 is longer than max_len1, intercepting a section from the end to make read1 equal to max_len1, which is 0 by default in this project, representing no limit;
-B, -max_len2 means, if read2 is longer than max_len2, intercepting a section from the end to make read2 equal to max_len2, which is 0 by default in this project, representing no limit;
(3) polyG tail trimming
-g, -trim_poly_g means intercepting polyG tail;
-poly_g_min_len means detecting the length of polyG at the tail of read, which is 10 by default in this project;
(4) polyX tail trimming
-x, -trim_poly_x means intercepting 3′ end polyX
-poly_x_min_len means detecting the length of polyX at the tail of read, which is 10 by default in this project;
(5) trimming of each read by quality value
-5, -cut front means moving a window from the 5′ end to the tail of the read, and removing bases whose average quality value in the window is less than a ‘<’ threshold, which is 20 in this project;
-3, -cut_tail means moving the window from a 3′ end value of the read to the head, and removing bases whose average quality value in the window is less than the ‘<’ threshold, which is 20 in this project;
-r, -cut_right means moving the window from the head to the tail of the read, if the average quality value of a window is less than the threshold, removing the base in the window and its right part, and stopping;
-W, -cut_window_size means sliding a window for filtering, which is similar to calculating kmer, 1 to 1000, 4 bases being used by default in this project;
-M means an average quality value of bases in the selected window, ranging from 1 to 36, which is Q20 by default in this project, if the average quality value in this area window is lower than 20, it being considered a low-quality area and removed;
-Q means controlling whether to remove low quality, wherein automatic removal is adopted by default in this project, that is, this parameter is not given;
-q means setting a low-quality standard, which is 20 in this project, that is, the quality value of less than 20 is considered a low-quality base, which is often called Q20;
-u means a percentage of low-quality bases, which is not to discard a piece of reads if it contains low-quality bases, but to set a certain proportion, wherein this project uses 40 by default, which means 40%, that is, 150 bp reads, the reads will be discarded if more than 60 low-quality bases are included, and will be discarded in pairs as long as one piece of reads does not meet the condition;
-n means filtering reads with too many N bases, wherein if the N base content is greater than n, this read/pair will be discarded, and this project uses 15, that is, more than 10% of N in 150 bp reads will be removed;
(6) length filtering option -L
-l is followed by a length value, reads less than this length will be discarded, and this project uses 80, that is, fragments below 80 bp will be filtered out;
(7) low complexity filtering -y, -low_complexity_filter means using low complexity filtering, where the definition of low complexity is a ratio of bases different from the next base (base [i]!=base [i+1]). -Y, -complexity_threshold, low complexity threshold (0 to 100), and this project uses 30 by default,
(8) related parameters of filtering result report
-j, -json means outputting a report file name in a json format (string [=fastp.json])
-h, -html means outputting a report file name in a html format, which may be viewed directly with a browser (string [=fastp.html])
-R, -report_title should be quoted with ‘or”, default is “fastp report” (string [=fastp report]);
(9) threads used in the filtering process
-w, -thread means the number of threads in use;
(10) filtered data: bowtie2 is used to align genomes before and after enzyme digestion, wherein the alignment parameters are -very-sensitive -L 30 -score-min L, -0.6, -0.2 -end-to-end;
(11) samtools view -F 4 is used to remove unaligned reads, convert to a bam format and sort according to physical positions of the genomes;
(12) after the sorting is completed, a samtools depth is used to obtain the depth of each position on the gene; and
(13) R software is used to compare the depth of each position between the two groups, Log 2 Coverage Ratio=log 2 (Depth of the sample after enzyme digestion/Depth of the sample before enzyme digestion); and draw a line graph with Log 2 Coverage Ratio; determine the position of nucleic acid double-strand break through the line graph, wherein in the line graph, a segment with Log 2 Coverage Ratio being less than 0.06 and an obvious valley is determined as a double-strand break segment, and the break position is determined as the lowest valley.

The above is only a preferred embodiment of the present invention. It should be pointed out that for those of ordinary skill in the art, some improvements and modifications can also be made without departing from the principle of the present invention, and these improvements and modifications should also be considered as the scope of protection of the present invention.

What is claimed is:

1. A method for identifying a digestion site of a nickase, comprising the following steps:
step 1, sequencing a sample before and after a nickase digestion to obtain first sample data before the nickase digestion and second sample data after the nickase digestion;
step 2, trimming and filtering the first sample data and the second sample data respectively to obtain first quality control data and second quality control data;
step 3, after aligning and sorting the first quality control data and the second quality control data, obtaining a sequencing depth of a base site; and
step 4, calculating a Log 2 Coverage Ratio value according to the sequencing depth of the base site, then making a line graph, and determining the digestion site of the nickase according to the Log 2 Coverage Ratio value and the line graph.

2. The method according to claim 1, wherein
the Log 2 Coverage Ratio value is: taking 2 as a base, a logarithm of a ratio of the sequencing depth of the base site after the nickase digestion to the sequencing depth of the base site before the nickase digestion.

3. The method according to claim 1, wherein a criterion for determining the digestion site of the nickase is: a lowest valley with the Log 2 Coverage Ratio value being less than 0.06 and an obvious valley fracture being the digestion site of the nickase.

4. The method according to claim 1, wherein in step 1, the sample is a double-stranded DNA sample.

5. The method according to claim 1, wherein in step 1, the sequencing is an mNGS next-generation sequencing.

6. The method according to claim 1, wherein in step 2, trimming parameters are set as follows: removing an adapter, setting -5 to 20, and setting -3 to 20.

7. The method according to claim 1, wherein in step 2, filtering parameters are set as follows: setting -q to 20, setting -n to 15, and setting -l to 80.

8. The method according to claim 1, wherein unaligned reads are configured to be removed after the aligning and before the sorting.

9. The method according to claim 1, wherein an alignment mode is an end-to-end mode, and parameters of the end-to-end mode are set as: very-sensitive, setting -L to 30, and setting -score-min to L, -0.6, -0.2.

10. The method according to claim 1, wherein the nickase is a restriction endonuclease configured to generate a single-strand nick in or adjacent to a specific DNA sequence.

11. The method according to claim 2, wherein a criterion for determining the digestion site of the nickase is: a lowest valley with the Log 2 Coverage Ratio value being less than 0.06 and an obvious valley fracture being the digestion site of the nickase.

12. The method according to claim 2, wherein in step 1, the sample is a double-stranded DNA sample.

13. The method according to claim 3, wherein in step 1, the sample is a double-stranded DNA sample.

14. The method according to claim 2, wherein in step 1, the sequencing is an mNGS next-generation sequencing.

15. The method according to claim 3, wherein in step 1, the sequencing is an mNGS next-generation sequencing.

16. The method according to claim 4, wherein in step 1, the sequencing is an mNGS next-generation sequencing.

17. The method according to claim 2, wherein in step 2, trimming parameters are set as follows: removing an adapter, setting -5 to 20, and setting -3 to 20.

18. The method according to claim 3, wherein in step 2, trimming parameters are set as follows: removing an adapter, setting -5 to 20, and setting -3 to 20.

19. The method according to claim 4, wherein in step 2, trimming parameters are set as follows: removing an adapter, setting -5 to 20, and setting -3 to 20.

20. The method according to claim 5, wherein in step 2, trimming parameters are set as follows: removing an adapter, setting -5 to 20, and setting -3 to 20.
